(19) United States
(12) Patent Application Publication
(10) Pub. No.: US 2013/0023736 A1
(43) Pub. Date: Jan. 24, 2013

(54) SYSTEMS FOR DRUG DELIVERY AND MONITORING

(71) Applicants: Stanley Dale Harpstead, Arden Hills, MN (US); Loren L. Barber, JR., Lake Elmo, MN (US)

(72) Inventors: Stanley Dale Harpstead, Arden Hills, MN (US); Loren L. Barber, JR., Lake Elmo, MN (US)

(21) Appl. No.: 13/187,658

(22) Filed: Jul. 21, 2011

(51) Int. Cl. A61B 5/00; A61P 37/02; A61K 49/00; A61K 39/395; A61K 31/437; A61K 9/00

(52) U.S. Cl. 600/300; 514/293; 424/484; 424/10.1; 424/130.1

(57) ABSTRACT

Films that comprise an active agent for delivery to a tissue. The films may include carboxyls that form a chemical association with the active agent and/or hydrophilic domains or particles that comprise the active agent.

### TECHNICAL FIELD

The technical field relates to delivery of biologically active agents to a tissue and monitoring a tissue response to the agents.

### BACKGROUND

Transdermal drug delivery has been a useful medical tool. Transdermal delivery systems have found significant clinical application in the delivery of systemic drugs for pain relief, hormone therapy, or treatment of nicotine addiction. More recent systems using chemical enhancers, non-cavitational ultrasound and iontophoresis have been used in clinical products. Some of these delivery systems target their effects to the stratum corneum layer of skin using microneedles, thermal ablation, microdermabrasion, electroporation or cavitational ultrasound.

### SUMMARY

Inventions are described herein that include embodiments for delivery of lipophilic, low doses of an active agent to target local dermal tissues. One embodiment for the delivery comprises chemical association of the active agent with free carboxylic acids of a film. The chemical association localizes the agent and may contribute to preserving its solubility at high concentrations that would otherwise lead to crystallization. An example of this association is a weak-acid to weak-base salt, with the carboxylic acid providing the weak acid. An alternative association is a charge-charge interaction, with the carboxylic acid providing a negative charge. Embodiments include chemical associations that exclude strong bonds such as covalent or ionic bonds.

One embodiment is a system for delivery of an active agent to a tissue comprising a film that isolates a tissue surface underneath the film, with the film comprising a continuous hydrophilic matrix and a discontinuous hydrophilic phase that comprises an active agent for eliciting a tissue response. The discontinuous hydrophilic phase may comprise a multitude of free carboxylic acid groups, which may further be provided at predetermined rations relative to the active agent.

Such a system, or others disclosed herein, may be used in a method of assessing a metastatic potential of a lesion comprising reviewing a plurality of images of an area of tissue that comprises the lesion, the images being taken over a period of time after application of an active-agent-releasing film to at least a portion of the area. At least a portion of the film may translucent and the reviewing may comprise comparing, in the images, tissue underneath the translucent portion. The agent may comprise an immune response modifier.

The films and systems may be incorporated within a kit for assessing a metastatic potential of a lesion comprising a plurality of the films comprising a translucent continuous hydrophilic matrix and an immune response modifier that elicits a tissue response visible through at least a portion of the film.

### BRIEF DESCRIPTION OF THE FIGURES

FIG. 1 is an illustration of a series of panels A, B, C, D, taken over a period of time showing images of lesions treated with a film that releases an immune response modifier that elicits a visible tissue reaction;

FIG. 2 depicts a film releasing an active agent and allowing entry of fluid and exit of water vapor; and

FIG. 3 depicts delivery of imiquimod from a film as compared to a conventional ointment-based delivery.

### DETAILED DESCRIPTION

Films may be prepared for delivery of an active agent to a tissue and evaluating a reaction of the tissue. The films isolate a tissue surface underneath the film and are translucent to allow visualization of the isolated tissue so as to assess the reaction of the tissue through the film. Films may be used to isolate an area from its surrounding environment. Films that are translucent provide opportunity to monitor the isolated area. Further, the traits in the film allow for control of interaction between the environment and the isolated area. One such method is through the control of moisture vapor transmission.

Films may, for instance, be used to deliver imiquimod. One method of delivery involves delivering the imiquimod to one or more lesions, regardless of whether or not they have been diagnosed as metastatic. The area under and/or around the film is monitored for signs of erythema or papules.

### Delivery of an Active Agent; Monitoring and Imaging

An application of the film is a system for delivery of a active agent to a tissue. In use, a film may be applied to a tissue or other area on a patient. The term tissue refers broadly to a location on a human or other animal. The tissues include those that are external (the epidermis), internal (dermis or organ), buccal, colonic, mucosal, and vaginal. The term film refers to a thin flexible sheet, with a hardness of less than about 80 A Durometer.

A film may be used in combination with ongoing imaging and monitoring. The images may be captured periodically. Comparison of the images provides a basis for diagnosis of cancerous lesions. A film may be prepared with one or more active agents and may further comprise an enhancer. The film is applied to a tissue or other site on a patient and periodically imaged. Further, the area around the film may also be monitored. Films may be prepared that are translucent so that an area under the film may be observed. The translucent film may be transparent, meaning having the property of transmitting rays of light so that bodies situated beyond or behind can be distinctly seen.

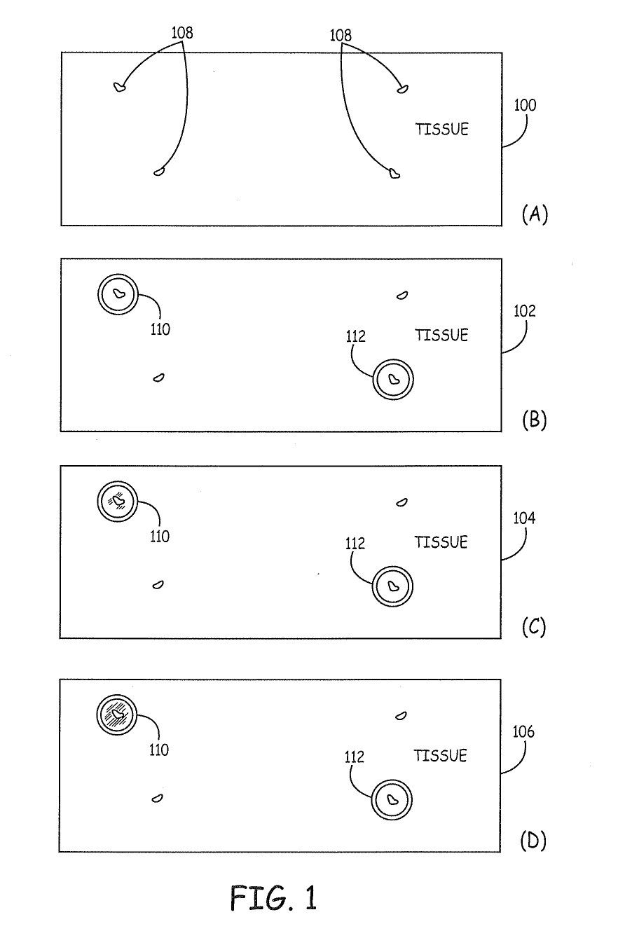

FIG. 1 depicts a series of images 100, 102, 104, 106 taken over a period of time, with a time interval or intervals separating the images. The images are of a living tissue that has a plurality of lesions 108. Two of the lesions receive films 110, 112, which are self-adhesive and translucent. The films are pre-loaded with the active agent imiquimod. Tissue under film 112 remains substantially unchanged over the period of time. Tissue under film 110 shows erythema in image 104, which is increased in image 106. The images are used for diagnosis of the presence or absence of cancer.

One embodiment provides for the film and/or area around the film to be imaged with a camera, e.g., selected from a group including a web camera, a mobile phone camera and a general purpose digital camera. The image is relayed to a medical practitioner such as a nurse or oncologist for evaluation. The image may be relayed by telephone, over the internet, with use of a personal digital assistant, an IPHONE, or other suitable means. The image may be relayed directly to the practitioner, e.g., by email, or to a central processing server or other location that provides a central location for retrieval by the practitioner and/or patient. After evaluation, the medical practitioner may return an analysis to the patient.

In some embodiments, software is provided that captures the image and automatically relays it with indicia of use, such as a patient identification, time, and date. Films may be provided with distinct identifiers so that a patient treated with multiple films can relay images that capture the distinct identifiers, so that the images can be analyzed efficiently. Further software processing provides for images associated with a particular film to be grouped together and arranged according to time. Software may be provided for a general-purpose computer or personal digital assistant (e.g., camera-ready cell phone, IPAD, IPHONE, notebook computer) that provides some or all of these functions.

A calibration standard may be used. The calibration standard provides a plurality of images for a user to compare to a treatment site. For instance, a calibration standard for redness may comprise a collection of shades or colors, e.g., a color strip, color palette, or wheel. Or a calibration standard may comprise a plurality of images of a lesion indicating a range from normal to highly reactive, with the images comprising erythema, and/or welling and/or papules. The calibration standard can be incorporated into the margins of the external facing surface of the film.

### Delivery of IRMs for Aid in Diagnosis

Films may, for instance, comprise an IRM agent. The film is applied to a patient's skin and the IRM, e.g., imiquimod, is delivered to the patient, regardless of whether or not the lesions have been diagnosed as metastatic. A user observes the film and changes to the tissue to monitor the effectiveness of the agent and progress of the treatment. Comparison of the images of an area over time provides a basis for diagnosis of cancerous lesions.

For example, a patient may apply an imiquimod-releasing film to a lesion, or other tissue, and capture a series of images of the area over time. The patient may transmit the images to a location for evaluation by a medical practitioner. The patient and/or practitioner may compare the images to a calibration standard. The standard may provide a basis for comparing, e.g., a color of the tissue, a swelling of a tissue (erythema), presence of papules, or a grading of margins of the lesions. The film may be a single film or one film in a set of films that are applied over a time series.

Lesions and other lesions that may be imaged and/or analyzed for diagnosis include basal cell carcinoma (BCC). BCC, a subtype of nonmelanoma skin cancer, is a malignancy arising from epidermal basal cells. BCC is a potentially fatal disease linked to sun exposure. Other lesions include squamous cell carcinoma (SCC). Primary cutaneous SCC is a malignant neoplasm of keratinizing epidermal cells. Commonly, SCC first appears as an ulcerated nodule or a superficial erosion on the skin or lower lip. Other lesions are actinic keratosis (AK). AKs are hyperkeratotic papules and plaques that occur on sun-exposed areas. Other lesions are those of lentigo maligna. Lesions may be located anywhere on the body including the lip, back, arm, back of the hand, leg, central face (e.g., the nose, the nasolabial fold, or the periorbital or perioral area), ears, scalp, and mucosal tissues. Methods of diagnosis with certain IRMs are described in US 20100180902.

Kits or systems may be prepared that comprise a plurality of films. Accordingly, a set of films may be prepared that comprise active films with an active agent and other inactive film(s) with no active agent. The films may be made so that they are indistinguishable from each other in their appearance (recognizing that they may have distinct numbers or other indicia). For instance, a pack of seven films may be prepared, with six of the films comprising an active agent and the seventh film being inactive. The films may be applied in a series having a predetermined order. The films in the series may have numbers indicating their order of application, be color-coded, or have other indicia on the films or associated packaging indicating their series order. Or the films may be packaged to present them in a series, for instance, by stacking them in a container that allows them to be removed one at a time, with the first in the series being removable first.

### Films

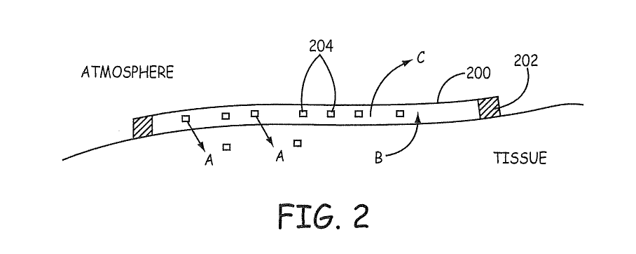

A first embodiment of the films provides a continuous hydrophilic matrix material comprising an active agent directly dispersed in the matrix and/or sequestered in domains dispersed in the matrix. As depicted in FIG. 2, for example, film 200 has an adhesive rim 202 and comprises active agent 204. Alternatively, the adhesive may be provided as a layer across some or all of the skin-contacting surface. In use on a tissue, the film releases active agent 204, as indicated by arrows A. Liquid from the tissue enters film 200 as indicated by arrow B. Vapor, which is largely water vapor, escapes from film 200 to the ambient gaseous surrounding, which is typically the atmosphere.

An embodiment of a film is a continuous hydrophilic matrix. The film matrix itself has covalent or non-covalent crosslinks so that it is insoluble in water, even though it is made with hydrophilic materials. Embodiments of hydrophilic matrices are set forth, for example, in U.S. Pat. Nos. 5,514,379, 5,874,500, 5,410,016, and US Pub. No. 2010/0033515, each of which is hereby incorporated by reference herein to the extent it does not contradict what is explicitly disclosed herein.

The matrix may further comprise a dispersed discontinuous phase, meaning a material distributed through the matrix, with the material being distinct from the matrix. The discontinuous phase may be, for instance, drops, particles, or self-assembling domains. An example of a self-assembling domain is a hydrophobic block or a micelle. An example of a drop is a liquid, sol, or gel. Examples of particles are liposomes, nanoparticles, microparticles, capsules, microcapsules, and solid beads. An embodiment of the discontinuous phase is a hydrophilic particle. The hydrophilic particle may be, e.g., a crosslinked particle that it is swellable in polar solvents. The term hydrophilic refers to a material that, when not in a crosslinked state, is soluble in water at a concentration of at least 1 g/100 mL in an aqueous solution. In the case of a crosslinked hydrophilic material, the material would swell in aqueous solutions and the crosslinks would prevent dissolution.

An embodiment of a film is a continuous hydrophilic matrix that comprises a plurality of discontinuous hydrophilic domains that are at least twice as swellable in aqueous media as the continuous hydrophilic matrix. Embodiments of the discontinuous phase are domains, e.g., particles, comprising crosslinked polyacrylic acid, crosslinked polyvinyl alcohols, crosslinked vinylpyrrolidinone, and crosslinked polyethylene glycols (PEGs, a term including polyethylene oxides with any termini unless otherwise indicated in the context). Embodiments of the crosslinked polymers, e.g., the polyacrylic acid, may include polymers with a very high molecular weight, e.g., more than 10 million, 100 million, or a billion. When these polymers are very high molecular weight versions they are not restricted to crosslinked structures. Lower molecular weights may also be used.

Embodiments of the continuous domains are polyvinyl alcohol, polyvinylpyrrolidinone, polyalkylenes, polyethylene glycols, hydrophilic polyurethanes, hydrophilic polyacrylates, and hydrophilic polymethacrylates.

Materials with pendant carboxylic acids may be used to form, or be part of, the continuous phase or discontinuous domains. These polymers may be purchased and reacted to make the films, or synthesized. For instance, methacrylic acid monomers may be reacted with other monomers to form polymers.

The discontinuous phase may be provided as a plurality of particles. Crosslinked hydrophilic polymer particles may be provided in the form of an at least partially neutralized crosslinked copolymer comprising a structure obtainable by a free radical polymerization product of at least one free-radically polymerizable carboxylic acid and a monomer comprising at least one of an alkyl or alkaryl(meth)acrylate, wherein the at least one alkyl or alkaryl(meth)acrylate has from 11 carbon atoms to 34 carbon atoms, and optionally additional co-monomers. As used herein, the term carboxylic acid encompasses the corresponding conjugate base (i.e., carboxylate). The free-radically polymerizable carboxylic acids have at least one carboxyl group and a polymerizable carbon-carbon double bond. Exemplary free-radically polymerizable carboxylic acids include, for instance, itaconic acid, methacrylic acid, acrylic acid, maleic acid, fumaric acid, salts of the foregoing, and mixtures thereof. The term (meth)acrylate refers to methacrylate and/or acrylate.

The copolymer that may be obtained by such a process refers to the structure of the copolymer rather than any particular method of preparing the copolymer. For example, the copolymer may be prepared using a monomer (e.g., maleic anhydride) that on hydrolysis (before or after co-polymerization) results in a free-radically polymerizable carboxylic acid. In order to ensure good swellability of the crosslinked copolymer, the acid content typically falls in a range of from about 40 percent to about 90 percent by weight (e.g., in a range of from 50 to 70 percent by weight) of the crosslinked copolymer, although acid content values outside this range may also by used.

The alkyl and alkaryl (meth)acrylates may have from about 11 carbon atoms to about 34 carbon atoms, and may be linear or branched. Examples of useful alkyl and alkaryl(meth)acrylates include octyl(meth)acrylate, isooctyl(meth)acrylate, octadecyl(meth)acrylate, tridecyl(meth)acrylate, and nonylphenyl acrylate. Optionally, additional co-monomers (e.g., (meth)acrylamide, butyl(meth)acrylate) may be included in the crosslinked copolymer.

Crosslinking can be accomplished by any suitable means, e.g., by inclusion of a monomer having multiple free-radically polymerizable groups (e.g., a polyfunctional monomer) in the monomer mixture prior to copolymerization, although other methods may be used. Useful polyfunctional monomers include, for example, vinyl ethers (e.g., pentaerythritol trivinyl ether, pentaerythritol tetravinyl ether, ethylene glycol divinyl ether), allyl ethers (e.g., pentaerythritol triallyl ether, pentaerythritol tetraallyl ether, ethylene glycol diallyl ether), and acrylates (e.g., 1,6-hexanediol diacrylate), and mixtures thereof. The amount of crosslinking desired typically determines the amount of polyfunctional monomer used. In order to ensure good swellability with water, the crosslink density should typically be kept at low level; for example, the value of M_{c} (i.e., the average molecular weight of segments between crosslinks) may be greater than about 1000 Daltons, preferably greater than about 2000 Daltons, and more preferably greater than about 3000 Daltons, or from 1000 to 1,000,000; artisans will immediately appreciate that all the ranges and values within the explicitly stated ranges are contemplated, e.g., from 1500 Daltons to 500,000 Daltons.

The discontinuous phase may be provided in a form (e.g., as particles) that is readily dispersible and water-swellable. This can be achieved, for instance, by the use of crosslinked-acrylic-copolymer having an average dry (substantially non-swelled) particle mean diameter in a range of from about 0.1 micrometer to about 10 micrometers, or from about 2 micrometers to about 7 micrometers, although larger and smaller particles may also be used; artisans will immediately appreciate that all the ranges and values within the explicitly stated ranges are contemplated.

Suitable crosslinked copolymers can be provided in particle sizes that are readily dispersible in a solvent solution, preferably without further cross linking to either similar particles or to other (e.g., heterogeneous) particles as the solvents evaporate. Particle sizes that are too large will tend to limit the strength of the second crosslinking copolymer; while particles that are too small will not create adequate voids in the course of dehydration, or in turn, swollen spaces during re-hydration. Applicant has discovered, inter glia, that particle mean diameter sizes in the range of about 0.1 micrometer to about 10 micrometer are preferred, particularly when dispersed within a continuous phase made using a crosslinked polyurethane copolymer. Without being bound to a particular theory, it would appear that the crosslinked polymer swells in the presence of various solvent solutions by attraction of solvent molecules, or through the interference with internal ionic bonding due to a change in pH, or the presence of either cations or anions in the solvent. Following the evaporation of the various solvents, it further appears that the crosslinked polyurethane polymer (phase) forms a stable film structure prior to the evaporation of the solvents in the discontinuous polymer leading to a potential for the discontinuous polymers to recapitulate their dimensions with restriction by the presence of the crosslinked polyurethane polymer. The swelling of the discontinuous phase (polymer) can be altered by altering the solvents in the solution while preserving the potential for the polyurethane to form a stable film structure. The amount of dispersed phase present in the continuous phase may be varied, e.g., from about 0.5 percent to about 55 percent w/w solid composition, i.e., as a percentage of the total weight of film components, and excluding solvents and excluding active agents. Artisans will immediately appreciate that all the ranges and values within the explicitly stated ranges are contemplated.

Compositions for making films typically involve dispersing or dissolving a small weight percent of a polymer in the solvent. For instance, hydrophilic polymer particles may be used in an aqueous solution phase, in an amount from about 0.5 percent by weight to about 3 percent by weight, e.g., as an emulsion, solution, or mixture, although higher and lower amounts may also be used. For example, a crosslinked-acrylic-copolymer particle may be present in a solution in a range of from about 1 percent by weight to about 2 percent w/w. These ranges are for guidance in preparation of suitable compositions and are exemplary; other concentrations may be used. The continuous phase may also be in the same solution phase, in an amount from 0.5 percent by weight to 3 percent by weight, such that after the solvents have evaporated the solid composition of the dispersed phase within the continuous phase would be about 50 percent w/w solid composition.

Additionally other matrices with free carboxyl groups can be made by grafting to other polymer backbones (e.g. cellulose, polyvinyl alcohol, polysaccharides) with chloroacetic acid in base to form the corresponding carboxymethyl cellulose, carboxymethyl polyvinyl alcohol, or carboxymethyl polysaccharide.

One process to prepare films involves mixing a first polymeric material with a second matrix material that forms a continuous matrix. The mixture is cast, molded, or otherwise distributed so that the continuous matrix is allowed to form around the first polymeric material. The resultant film has a continuous matrix with discontinuous subdomains dispersed throughout it. The discontinuous domain may be, for instance, crosslinked polymers or particles (nanoparticles or microparticles). The continuous domain may be, for instance, a thermoset, thermoplastic, or crosslinked polymer. The first polymeric material may comprise one or more precursors or polymers that form the continuous domain, e.g., precursors to a polyurethane, a resin, a rubber, free radical polymerizable polymers, or precursors that chemically react.

Embodiments of a film include a single-layer film or a film with a plurality of layers wherein at least one of the layers include a polyurethane continuous matrix and a plurality of discontinuous domains, e.g., a crosslinked polyacrylic acid. Examples of polyurethanes for either phase are set forth in, e.g., U.S. Pat. Nos. 6,734,273, 5,428,123 and U.S. Pat. Pub. No. 2007/0112165, the disclosures of which are hereby incorporated herein by reference, with the instant specification controlling in the case of conflict.

The degree of crosslinking, distance between crosslinks, and length of a crosslinker can affect swelling. Accordingly, for example, PEG particles may be present in a PEG matrix and still exhibit twice as much swelling. Swelling is a term that refers to the amount of swelling of the dried material as compared to its swollen weight in the presence of an excess of distilled water at room temperature. Other ratios of swelling may be used, e.g., from 1:1 to 100:1 of discontinuous domain: continuous domain, with “twice” referring to a 2:1 ratio; artisans will immediately appreciate that all the ranges and values within the explicitly stated ranges are contemplated, for instance, between 2:1 and 10:1, or at least 3:1.

The film may be provided with one or more of an active agents. These may be in the continuous phase and/or discontinuous phase. For instance, collections of particles may be prepared that each comprise different agent and particles from a plurality of the collections may be added to the film. Or a first active agent may be in a continuous phase and a second agent in a discontinuous phase.

For example, a film may have a first agent in a polyacrylic acid particle or other subdomain within a matrix and in that same matrix may have a second agent in a second polyacrylic acid particle or other subdomain. A method to make such a film is by first loading the polyacrylic acid particle with the agent (or incorporating the agent at the time of formation) and then blending the particle into a solution with polyurethane or other hydrophilic polymers to form the film.

Films comprising matrices with pendant carboxylic acids have been found to exhibit unpredictable results that are unexpected and surprising. In brief, as detailed in Example 4, more active agent (imiquimod) was released from films with a low amount of agent compared to films with higher loadings. Specifically, films loaded with less imiquimod delivered more imiquimod to the tissue compared to films with higher imiquimod loadings. Without being bound to a particular theory, the higher loadings are believed to result in crystallization of the agent and a consequent lowered release rate. A further non-binding theory of operation is that the weak base-weak acid interactions between nitrogen groups of the agent and carboxylic acids is believed to prevent crystallization up to a certain point, after which crystallization effects dominate. The carboxylic acid provides the weak acid so that an active agent that is a weak base can be stabilized and resist crystallization. As a corollary, more than one carboxyl may bind an active agent to increase stabilization. As another corollary, regions with a high density of carboxyls tend to exert more favorable stabilization.

Accordingly, a suitable ratio of active agent molecules to carboxylic acids groups in a film or a domain of a film may be 1:n (i.e., a ratio of one molecule of imiquimod to n carboxylic acid groups), with n being between about 3 and about 200; artisans will immediately appreciate that all the ranges and values within the explicitly stated ranges are contemplated, e.g., n between 5 and 50, or 10 and 25. The ratio may also be viewed in terms of molecular weight, as in the Examples, wherein the equivalent weight of a COOH group in polyacrylic acid is about 76 MW and the agent (imiquimod) has a MW of 240.31. Relative molecular weights of 1:1 or 1:10 (agent:carboxyl) are suitable, as well as 1:m (agent:carboxyl) with m being between 1 and 500; artisans will immediately appreciate that all the ranges and values of m within the explicitly stated ranges are contemplated. Moreover, some embodiments involve choosing polymers that comprise a minimum density of pendant carboxylic acid groups, e.g., polymers comprising a pendant carboxylic acids with no more than about 2 to 50 carbon bonds of separation on the polymer backbone between the carboxylic acid groups: for instance, a polyacrylic acid or derivative thereof. Artisans will immediately appreciate that all the ranges and values within the explicitly stated ranges are contemplated, e.g., between 2 and 10 carbon bonds. The description in terms of carbon bonds refers to a distance; polymers with backbones besides C—C bonds may be used.

The films are flexible and thin. Flexibility contributes to ease of use for the end-user that wears the film. Thinness contributes to flexibility and assists in keeping the film in place on an exposed tissue, with a thin film being less likely to be displaced in use on a patient. The thinness also impacts drug release properties. The films may be made to be surprisingly thin, with a thickness of less than 250 microns; artisans will immediately appreciate that all the ranges and values within the explicitly stated ranges are contemplated, e.g., less than about 100 or about 200 microns, or between about 10 and about 100 microns. As demonstrated herein, drug loadings may be high, with small volumes being practivable, e.g., volumes of no more than 5 or 10 cubic centimeters (cc); artisans will immediately appreciate that all the ranges and values within the explicitly stated ranges are contemplated, e.g., between 0.1 and 5 cc, or between 1 and 10 cc.

Films may be made with self-adhesive properties. Such films may also be prepared with a pressure-sensitive adhesive. The adhesive may be applied to all or a portion of a side of a film. The active agent and carrier polymers can be incorporated into the adhesive layer. In a first embodiment, the adhesive covers one side of the film. In a second embodiment, the adhesive is at a periphery of the film. The film may further be attached to a member that receives the adhesive, e.g., a cardboard or elastic member that is tied to the film. The film may be prepared with a backing member that is removable by a user to expose the adhesive at the time of use. The film may be prepared in a sterile package.

### Active Agents

A film may comprise an active agent. An active agent refers to a biologically active compound. A film may have one or more agents. Examples of therapeutic compounds are antibiotics, antihistamines and decongestants, anti-inflammatory agents, antiparasitics, antivirals, local anesthetics, antifungal agents, amoebicidal agents, trichomonocidal agents, analgesics, antiarthritis agents, antiasthmatics, antidepressants, antidiabetics, antineoplastics, antipsychotics, neuroleptics, antihypertensives, antidepressants, hypnotics, sedatives, anxyolitic energizers, anticonvulsants, immune suppression agents, antiparkinson agents, anti-platelet agents, anti-cancer agents, muscle relaxant agents, antimalarials, blood modifiers, hormonal agents, contraceptives, sympathomimetics, diuretics, hypoglycemics, anti-coagulation agents, ophthalmics, anti-cell proliferation agents, electrolytes, diagnostic agents and cardiovascular drugs.

Antibodies may also serve as active agents. The term antibody is used broadly herein and includes monoclonal and polyclonal antibodies, as well as antibody fragments. Further, imiquimod or other immune response modifiers (IRMs) may be used as active agents. Further, biological segments intended to serve as an antigen may be delivered in combination with an IRM or independently. Moreover, adjuvants may be delivered with an IRM and/or an antigen. The antigens may be used to promote innate production of antibodies in a recipient.

The films may further include an enhancer, e.g., hydrophobic enhancer. The term enhancer refers to an agent known to enhance penetration of an active agent into the stratum corneum. Many effective chemical enhancers disrupt the stratum corneum by inserting amphiphilic molecules to disorganize molecular packing or by extracting lipids to create lipid packing defects. Various chemical enhancers have been studied, including lactic acid, AZONE (1-dodecylazacycloheptane-2-one) and SEPA (2-n-nonyl-1,3 dioxolane). The term hydrophobic enhancer refers to long chain alkyls of at least 10 carbon groups. Examples of hydrophobic enhancers include oleic acid, stearic acid, isostearic acid, palmitic acid, and fatty acids.

### Immune Response Modifiers

An embodiment of a active agent is an immune response modifier (IRM). IRMs are described, for instance, in US Pub. 2004/0265351 which is hereby incorporated herein by reference for all purposes; in case of conflict, the specification is controlling. IRMs such as imiquimod, a small molecule, imidazoquinoline IRM, marketed as ALDARA (3M Pharmaceuticals, St. Paul, Minn.) have been shown to be useful for the therapeutic treatment of warts, as well as certain cancerous or pre-cancerous lesions (for example, Geisse et al., J. Am. Acad. Dermatol., 47(3): 390-398 (2002); Shumack et al., Arch. Dermatol., 138: 1163-1171 (2002); U.S. Pat. No. 5,238,944 and International Publication No. WO 03/045391.

IRMs are compounds that act on the immune system by inducing and/or suppressing cytokine biosynthesis. In particular, certain IRM compounds effect their immunostimulatory activity by inducing the production and secretion of cytokines such as, e.g., Type I interferons, TNF-alpha., IL-1, IL-6, IL-8, IL-10, IL-12, IP-10, MIP-1, MIP-3, and/or MCP-1, and can also inhibit production and secretion of certain TH-2 cytokines, such as IL-4 and IL-5. Some IRM compounds are said to suppress IL-1 and TNF (U.S. Pat. No. 6,518,265).

IRM compounds include small molecule IRMs, which are relatively small organic compounds (molecular weight under about 1000 Daltons). Although not bound by any single theory of activity, some IRMs are known to be agonists of at least one Toll-like receptor (TLR). IRM compounds include a 2-aminopyridine fused to a five-membered nitrogen-containing heterocyclic ring. Examples of classes of small molecule IRM compounds include, e.g., derivatives of imidazoquinoline amines including but not limited to amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, and thioether substituted imidazoquinoline amines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, and thioether substituted tetrahydroimidazoquinoline amines; imidazopyridine amines including but not limited to amide substituted imidazopyridines, sulfonamido substituted imidazopyridines, and urea substituted imidazopyridines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; and thiazolonaphthyridine amines, such as those disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,545,016; 6,545,017; 6,558,951; and 6,573,273; European Patent 0 394 026; U.S. Patent Publication No. 2002/0055517; and International Patent Publication Nos. WO 01/74343; WO 02/46188; WO 02/46189; WO 02/46190; WO 02/46191; WO 02/46192; WO 02/46193; WO 02/46749; WO 02/102377; WO 03/020889; WO 03/043572 and WO 03/045391. Examples of small molecule IRMs that include a 4-aminopyrimidine fused to a five-membered nitrogen-containing heterocyclic ring include adenine derivatives (such as those described in U.S. Pat. Nos. 6,376,501; 6,028,076 and 6,329,381; and in WO 02/08595). IRM compounds include resiquimod.

Other IRM compounds include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. CpG7909 is a specific example. Other IRM nucleotide sequences lack CpG and are described, for example, in International Patent Publication No. WO 00/75304.

### EXAMPLES

### Example 1

### Film Fabrication

(A) Solutions for the Tissue Adhesive Layer (TAL; layer intended to face the skin)
a. Wetting of PEMULEN TR-2 poly acrylic acid (PAA): Combine 3 parts PEMULEN TR-2 NF (Noveon), 27 parts propylene glycol monomethyl ether acetate (PMA, and mix vigorously to remove “clumps” to wet the TR2
b. Combining PEMULEN TR-2 and hydrophilic polyurethane (PU(H)): Combine 20 parts tetrahydrofuran (THF), 3 parts acetone, 4 parts PU(H) 12.5% solution dissolved in THF, 4 parts TR-2 wetted 10% in PMA, 0.5 parts ethanol (EtOH), 0.5 parts water (diH₂O) and shake vigorously and place on rotating mixer for 24 hours

### (B) Solutions for the Agent Carrying Layer (ACL) in EtOH

a. Prepare swollen TR2-PAA: Combine 60 parts ethanol (EtOH), 12 parts THF, 16.5 parts TR2 (10% wetted in PMA), shake vigorously and place on rotating mixer
b. Prepare ACL solution (combining PU(H) and swollen TR2-PAA): Combine 12 parts THF, 6 parts PU(H) 12.5% solution dissolved in THF; 38 parts swollen TR-2 and place on rotating mixer until used.
   (C) PU(H) in THF (Used for the external surface)—6.25%: Combine 1 part THF, 1 part PU(H) 12.5% dissolved in THF, shake vigorously and place on rotating mixer until used.
   (D) Imiquimod in ACL solution: Combine 14.1312 g ACL and 37.7 mg of imiquimod (Sigma Aldrich), sonicate, place on rotating mixer until used. Provides a ratio of imiquimod_{eq} to PAA_{eq} of 1:15 (i.e., a ratio of one molecule of imiquimod to 15 carboxylic acid groups). Other ratios may similarly be prepared.
   (E) Place sheets polyethylene (PE) extrusion over a glass plate as a release liner. Place about 8 g of solution of the Tissue Adhesive Layer (TAL) solution on the top edge of one of the PE films and create a film coat using a 12 coil Meyer coating rod. Allow each coat to dry before proceeding.
   (F) On a film containing the TAL, then add the Agent Carrying Layer (ACL) imiquimod in a 1:15 ratio. On the upper half of the film, spots of 100 μl ACL/imiquimod solution was pipetted on the film and allowed to dry. This process was repeated twice for a total application of 200 μl. The calculated imiquimod amount in these spots was 171 μg/cm² of imiquimod. On the lower half of the film, a coat of ACL/imiquimod solution was scrape-coated with a 12 coil (thread per inch) Meyer rod (8 mil coating thickness) resulting in a calculated amount of imiquimod of 44 μg/cm2. On a second sheet of PE release liner, the 6.25% solution was spread using the 12 coil Meyer coating rod. The first coat included 10 g of 6.25% PU(H) solution. A second coat of 13.4 g of the same material was spread over the first coat. After 10 minutes this film was pressed on top of the drug coated layers and the two layers were allowed to fuse. The film was placed into the oven overnight to drive out any residual solvents. After the film is dried, spots may be selected for elution testing.

### Example 2

### Delivery of Hydrophobic Agent

A first group of samples (Group 1) were prepared from the films of Example 1 by using the area that was formed by scrape-coating the ACL/Imiquimod solution; these films had a driving force of about 44 μg imiquimod per cm². A second group of samples (Group 2) were prepared from the films of Example 1 by using the spots where the ACL/Imiquimod was placed by dropper; these films had a driving force of about 342 μg imiquimod/cm². Another group of four samples (Group 3) were made from 5% imiquimod cream (Nycomed, Fougera) placed between two 0.45 μm polycarbonate membranes; these films had an average driving force of about 263 μg imiquimod. The samples were punched out of the film and placed in the diffusion fixtures.

The receiving fluid on each side of the membrane was physiological phosphate buffered saline (PBS) (3 ml). Tests were run for 24 hours at 32° C. on a rotating table. Samples were collected from both sides of the film. The films were loaded so that the side intended to face the tissue (skin-side) is at the back and the film side that is intended to be away from the tissue (external-side) is in the front. Samples (200 μl) were collected at T0, T1-hour, T2-hour, T6-hour, and after 24 hours. Samples were placed directly into 300 μl HPLC vials and stored at 4° C. until assayed by HPLC.

Group 1 films delivered a cumulative average of 33.6 μg of imiquimod to both sides of the membrane over 24 hours (about 76% of the driving load). During the course of the experiment (hours 1, 2, and 6) the imiquimod delivered to the skin-side was about twice the imiquimod delivered to the external-side. Significant imiquimod was released in the first hour (about 52%). This result indicates that the drug was stabilized in the film, which enabled quick and full release of the dissolved drug. These results were consistent with what was seen in other experiments (not shown) used to develop the HPLC methods.

The cumulative average release of imiquimod from Group 2 films was 159.1 μg or 46% of the initial load. Again the amount released to the skin-side was about twice the imiquimod delivered to the external-side. A significant imiquimod was released in the first hour (about 7.8% of the driving load).

The 5% imiquimod cream samples of Group 3 released 20.1 μg into the PBS over 24 hours. The percent of driving load for these samples averaged 7.7%, which was less than seen in the films above. There was no evidence of imiquimod diffusing in a preferred direction in these samples.

This Example demonstrates the ability to release imiquimod by scrape-coat formation of a drug-containing layer. It further demonstrated that an external layer of the films may be provided that favors elution of imiquimod preferentially in the direction of the skin. Finally, it confirms that the cream solution does not allow for easy release of imiquimod into aqueous solutions.

### Example 3

### Preparation of Films

Films were prepared as described in Example 1, with exceptions as described. Step (B)(a) Swollen TR2-PAA was made with 25 parts ethanol (EtOH) and 25 parts TR2 (10% wetted in PMA). Step B(b) ACL solution (combining PU(H) and swollen TR2-PAA) was made with 3 parts THF, 12.4 parts PU(H) 12.5% solution dissolved in THF, and 46 parts swollen TR-2. An amount of 35.7 mg of imiquimod was used to make a ratio of imiquimod_{eq} to PAA_{eq} carboxylic acid groups=1:28 or the imiquimod concentration was increased to achieve a desired ratio. The imiquimod was diluted in EtOH and THF.

About 10 g of solution of the TAL solution was spread using the 12 coil Meyer coating rod. On this film containing the TAL, the Agent Carrying Layer loaded with imiquimod in a 1:28 ratio. A first coat of ACL/imiquimod solution was scrape-coated resulting in a calculated amount of imiquimod of 40.157 μng/cm². A second coat of ACL/imiquimod solution was scraped resulting in a calculated amount of imiquimod of 40.157 μg/cm². The cumulative area with two coats contained ˜80 μg/cm². A third coat of ACL/imiquimod solution was scraped resulting in a calculated amount of imiquimod of 40.157 μg/cm². The cumulative area with three coats contained about 120 μg/cm².

### Example 4

### Delivery of Imiquimod from Films Compared to Commercial Cream

This Experiment demonstrates that imiquimod can be delivered effectively from films and supplied into and through the tissue even when utilizing a driving quantity of drug that is low compared to conventional cream-based delivery. The films of Example 3 were used; these contained 120 μg/cm², a PAA_{eq} to imiquimod_{eq} ratio of 28:1; and utilized three sequential layers that contained the drug.

In brief, tissue samples were prepared from frozen porcine skin and placed into PBS solution and stored overnight at 4° C. No tissue was used more than 48 hours after thawing. 6 well plates were laid out such that each plate contained 3 samples of film, two samples with 5% imiquimod cream (Nycomed, Fougera) and 1 negative control. Tests were run for 24 hours at 32° C. on a rotating table. Samples were collected after 24 hours; recovery of PBS media; tape stripping of stratum corneum (10× each site); and homogenization of ½″ tissue plug. Samples were stored at 4° C. until assayed with HPLC method.

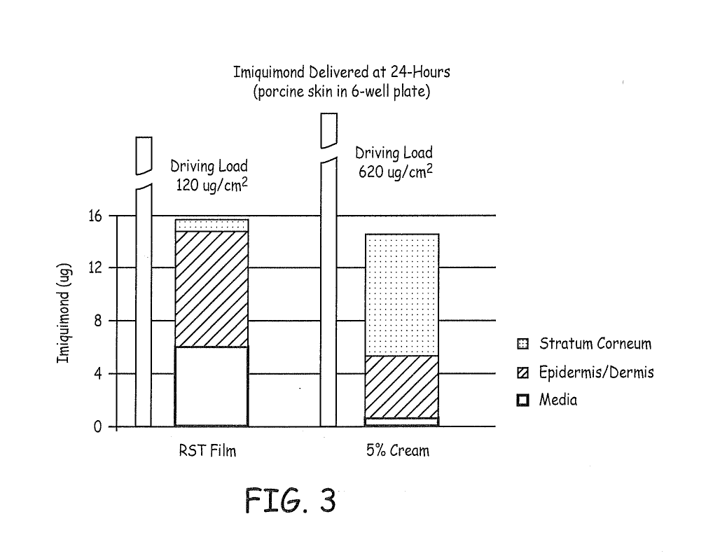

In this Example, the driving force per cm² for the film was about 120 μg and the comparable driving force for the imiquimod cream averaged 620 μg (based on weight applied). FIG. 3 shows that the film was effective for delivery. The film delivered 0.77 μg±0.11 μg of imiquimod to the stratum corneum (mean±SEM); 8.84±1.94 μg into the epidermal/dermal tissue and 6.11±2.01 μg into the PBS media within 24 hours. The total imiquimod delivered was 14.47±3.62 μg or 12.1% of the loaded imiquimod per cm².

The 5% imiquimod cream delivered 9.15 μg±1.77 μg of imiquimod to the stratum corneum (mean±SEM); 4.77±1.00 μg into the epidermal/dermal tissue and 0.73±0.44 μg into the PBS media within 24 hours. The total imiquimod delivered was 14.64±2.09 μg or 2.4% of the loaded imiquimod per cm². A comparable amount of imiquimod can be delivered from the film reaching into and through the tissue even with utilizing a lower driving quantity of drug.

The HPLC assay of this experiment included several elution samples taken from the films that had been soaked directly in PBS. In summary, in an area of the film that only received one coating of the drug layer, the amount eluted (n=3) was 18.7 μg per cm². When compared to the expected initial loading of 40 μg this represents a recovery of 46.8%. In the area of the film that received two coats of the drug layer the amount eluted (n=3) was 45.7 μg per cm². When compared to the expected initial loading of 80 μg this represents a recovery of 57.1%. In the area of the film that received three coats of the drug layer [these are equivalent to the film samples described above] the amount eluted (n=3) was 64.7 μg per cm². When compared to the expected initial loading of 120 μg this represents a recovery of 53.9%.

### Example 5

### Release of Imiquimod

This experiment describes the effect of the imiquimod to PAA ratio on the release of imiquimod and ability to form clear films. The imiquimod to PAA ratio was varied and two different coating thicknesses were used for comparison of these methods. Disc samples with a ¼″ diameter were soaked in PBS for 24 hours and the eluted imiquimod measured.

Films were prepared according to the methods of the Examples above and as follows:

| | Parts | |
|---|---|---|
| PMA | 6.38 | 11.39% |
| THF | 22.40 | 40.00% |
| EtOH | 25.76 | 46.00% |
| diH2O | 0.00 | 0.00% |
| PU(H) | 0.75 | 1.34% |
| PAA | 0.71 | 1.27% |
| Total | 56.00 | 100.0% |

Imiquimod was added to aliquots of the above solution and sonicated. The polar equivalent ratios were: 1 imiquimod molecule to 10 carboxylic acid groups in PAA (1:10); 1 imiquimod molecule to 15 carboxylic acid groups in PAA (1:15); and 1 imiquimod molecule to 20 carboxylic acid groups in PAA (1:20). Films were prepared with a thickness of 8.2 mil (12 coils) or 13.1 mil (7 coils) and completely dried in a 37° C. oven before tested. ¼″ pieces were punched out of the films and placed in 1 ml PBS in sealed microtubes; the microtubes were placed on a rotating table in a 32° C. cabinet. After 24 hours the physiological buffer (phosphate buffered saline, PBS) was filtered through 0.45 micron filters and placed into high pressure liquid chromatography (HPLC) vials for assay.

The calculated potential driving quantity of imiquimod per ½″ diameter film disk was:
1:10; 12 coils per inch→20.66 μg
1:10; 7 coils per inch→33.01 μg
1:15; 12 coils per inch→13.77 μg
1:15; 7 coils per inch→22.00 μg
1:20; 12 coils per inch→10.33 μg
1:20; 7 coils per inch→16.50 μg

The average amount (μg) of imiquimod recovered from four samples of each ¼″ disk after 24 hours of elution was:

Coils per Inch

| | 7 | 12 |
|---|---|---|
| 1:10 | 2.51 μg | 2.54 μg |
| 1:15 | 2.62 μg | 2.47 μg |
| 1:20 | 2.96 μg | 2.61 μg |

Surprisingly, in spite of the greater initial driving quantity of imiquimod in each film that amount recovered was inversely correlated. Furthermore, the coating thickness was also inversely correlated with the ability to elute imiquimod. Below is the percentage recovered of the initial loaded quantities.

Coils per Inch

| | 7 | 12 |
|---|---|---|
| 1:10 | 7.6% | 12.3% |
| 1:15 | 11.9% | 17.9% |
| 1:20 | 18.0% | 25.3% |

The greatest percentage eluted was in the films formulated out of the lower imiquimod to PAA ratios in the thinner layers.

All patents, patent applications, and publications referenced herein are hereby incorporated herein by reference for all purposes; in case of conflict, the instant specification is controlling.

1. A system for delivery of an active agent to a tissue comprising:
a film that isolates a tissue surface underneath the film, with the film comprising a continuous hydrophilic matrix and an active agent for eliciting a tissue response wherein the film comprises carboxylic acid groups that form a weak acid-weak base salt with the active agent.

2. The system of claim 1 wherein the film further comprises a discontinuous hydrophilic phase that comprises the carboxylic acid groups.

3. The system of claim 1 wherein the active agent comprises an immune response modifier.

4. The system of claim 3 wherein the immune response modifier comprises imiquimod.

5. The system of claim 4 wherein the film comprises carboxylic acids that form a weak acid-weak base salt with the imiquimod.

6. The system of claim 5 wherein the ratio of molecular imiquimod to carboxylic acid groups is between 1:5 and 1:50 imiquimod:carboxylic acid.

7. The system of claim 1 wherein the tissue response comprises erythema and/or a papule.

8. The system of claim 1 wherein the film provides passage of water from the tissue into the film and transmission of water vapor out of the film to ambient air or other gas.

9. The system of claim 1 further comprising a skin penetration enhancer and/or a second active agent.

10. The system of claim 1 wherein the film further comprises an adhesive surface for adherence to the tissue.

11. The system of claim 10 wherein the adhesive surface also comprises the active agent.

12. The system of claim 1 wherein the active agent is chosen from the group consisting of peptides, antibodies, and drugs.

13. The system of claim 1 wherein the film has a thickness of no more than about 100 μm.

14. The system of claim 13 wherein the film has a volume, when hydrated in aqueous medium, of no more than about 5 cubic centimeters.

15. The system of claim 1 wherein the continuous hydrophilic matrix comprises a polyurethane.

16. The system of claim 1 wherein the continuous hydrophilic matrix comprises a member chosen from the group consisting of polyvinyl alcohol, vinylpyrrolidinone, polyalkylenes, polyethylene glycols, hydrophilic polyacrylates, and hydrophilic polymethacrylates.

17. The system of claim 1 wherein the film further comprises a plurality of discontinuous hydrophilic domains dispersed in the continuous hydrophilic matrix.

18. The system of claim 17 wherein the hydrophilic domains comprise particles comprising crosslinked hydrophilic polymers.

19. The system of claim 17 wherein the continuous hydrophilic matrix comprises the active agent.

20. The system of claim 17 wherein the discontinuous domains comprise the active agent.

21. The system of claim 17 wherein the discontinuous domains are at least twice as swellable in aqueous media as continuous hydrophilic matrix.

22. The system of claim 1 further comprising a plurality or microparticles and/or nanoparticles that comprise the active agent.

23. The system of claim 1 with the film further comprising a calibration standard on an external facing surface of the film for comparing a plurality of images of the tissue.

24. The system of claim 1 wherein the films comprise an identifier on an external facing surface of the films for distinguishing the films relative to each other in images of the films.

25. A method of assessing a metastatic potential of a lesion comprising:
reviewing a plurality of images of an area of tissue that comprises the lesion, the images being taken over a period of time after application of an imiquimod-releasing film to at least a portion of the area, wherein at least a portion of the film is translucent and the reviewing comprises comparing, in the images, tissue underneath the translucent portion.

26. The method of claim 25 wherein the reviewing comprises comparing, in the images, erythema and/or papules of the tissue around the lesion.

27. The method of claim 25 wherein the images are received from a patient treated with the film.

28. The method of claim 25 wherein the plurality of images comprise a first image taken on a day when the film is applied to the tissue and a second image taken on a day that is more than four days after the first image.

29. The method of claim 25 wherein the film comprises a continuous hydrophilic matrix and a plurality of discontinuous hydrophilic domains that comprise the imiquimod.

30. A kit for assessing a metastatic potential of a lesion comprising:
a plurality of films comprising a translucent continuous hydrophilic matrix and an immune response modifier that elicits a tissue response visible through at least a portion of the film.

31. The kit of claim 30 wherein the immune response modifier comprises imiquimod.

32. The kit of claim 30 wherein one or more of the films has a thickness of no more than about 100 μm and a volume, when hydrated in aqueous medium, of no more than about 5 cubic centimeters.

33. The kit of claim 30 further comprising an inactive film that does not deliver an active agent, with the inactive film being provided as a member of a series that presents all of the films in an order of application to the tissue.

34. The kit of claim 33 wherein the films are numbered in a series or wherein the films are stacked in a container to thereby present the series.

35. The kit of claim 30 further comprising a calibration standard for comparing a plurality of images of the tissue.

36. The kit of claim 30 wherein the films comprise an identifier for distinguishing the films relative to each other in images of the films.

37. The kit of claim 30 wherein the immune response modifier comprises imiquimod and the film comprises carboxylic acids that form a weak acid-weak base salt with the imiquimod.
